(19) United States
(12) Patent Application Publication
(10) Pub. No.: US 2023/0258795 A1
(43) Pub. Date: Aug. 17, 2023

(54) INFORMATION PROCESSING DEVICE, INFORMATION PROCESSING METHOD, AND PROGRAM

(71) Applicant: TOYOTA JIDOSHA KABUSHIKI KAISHA, Toyota-shi (JP)

(72) Inventors: Toyokazu NAKASHIMA, Nagoya-shi (JP); Shunsuke SAGARA, Nissin-shi (JP)

(21) Appl. No.: 18/055,459

(22) Filed: Nov. 15, 2022

(30) Foreign Application Priority Data
Feb. 15, 2022 (JP) 2022-021656

(51) Int. Cl. G01S 13/90; H04W 4/44; G01B 17/08

(52) U.S. Cl. CPC G01S 13/90; H04W 4/44; G01B 17/08

(57) ABSTRACT

The information processing device includes a control unit. The control unit is configured to identify a portion where the predetermined unevenness is generated based on the information on the unevenness of the road surface acquired from the vehicle, and to provide data in which the portion where the predetermined unevenness is generated is observed by the artificial satellite.

### CROSS-REFERENCE TO RELATED APPLICATION

This application claims priority to Japanese Patent Application No. 2022-021656 filed on Feb. 15, 2022, incorporated herein by reference in its entirety.

### BACKGROUND

### 1. Technical Field

The present disclosure relates to an information processing device, an information processing method, and a program.

### 2. Description of Related Art

There is a technique of determining unevenness of a road surface based on a rotational speed of a wheel (for example, Japanese Unexamined Patent Application Publication No. 2019-49952 (JP 2019-49952 A)).

### SUMMARY

An object of the present disclosure is to provide a technique capable of eliminating the time and effort associated with inspection of an actual road surface.

An aspect of the present disclosure is an information processing device including a control unit that: identifies a portion where predetermined unevenness is generated based on information on unevenness of a road surface, the information being acquired from a vehicle; and provides data observed by an artificial satellite, the data being data of the portion where the predetermined unevenness is generated.

Another aspect of the present disclosure is an information processing method including: identifying, by an information processing device, a portion where predetermined unevenness is generated based on information on unevenness of a road surface, the information being acquired from a vehicle; and providing, by the information processing device, data observed by an artificial satellite, the data being data of the portion where the predetermined unevenness is generated.

Still another aspect of the present disclosure is a program that causes a computer to execute the above-described information processing method, or a computer-readable storage medium storing the program in a non-transitory manner.

According to the present disclosure, it is possible to save time and effort associated with inspection of an actual road surface.

### BRIEF DESCRIPTION OF THE DRAWINGS

Features, advantages, and technical and industrial significance of exemplary embodiments of the present disclosure will be described below with reference to the accompanying drawings, in which like signs denote like elements, and wherein:

FIG. 1 is a diagram illustrating a configuration example of an information processing system according to an embodiment;

FIG. 2A is a diagram illustrating a configuration of an information processing device;

FIG. 2B is a diagram illustrating a configuration of a terminal;

FIG. 3 is a diagram illustrating a configuration example of an in-vehicle terminal;

FIG. 4 is a sequence diagram illustrating an operation example of the embodiment; and

FIG. 5 is a flowchart illustrating an example of the unevenness determination process.

### DETAILED DESCRIPTION OF EMBODIMENTS

When the road condition deteriorates to such an extent that the road condition affects the traveling of the vehicle due to cracking, depression, or elevation of asphalt on the road, the road manager repairs the road. On the other hand, when the number of roads to be managed and the installation area are large, it is difficult to check all of the road conditions by inspection (visual inspection). For this reason, for example, it is conceivable to detect the unevenness of the road surface on the basis of the rotational speed of the wheels of the vehicle.

However, the unevenness of the road surface detected based on the rotational speed of the wheel is only an estimation result. That is, it is unclear whether such unevenness actually occurs on the road surface or whether the unevenness requires repair of the road surface. Therefore, it is considered that the administrator of the road needs to visit the location where the unevenness is detected and actually inspect the location. However, this inspection was troublesome. In addition, when it was found that the inspection was erroneous detection, the inspection may be wasted.

Hereinafter, an information processing device, an information processing method, and a program capable of solving the above-described problem will be described with reference to the drawings. The configurations of the embodiment are shown as examples.

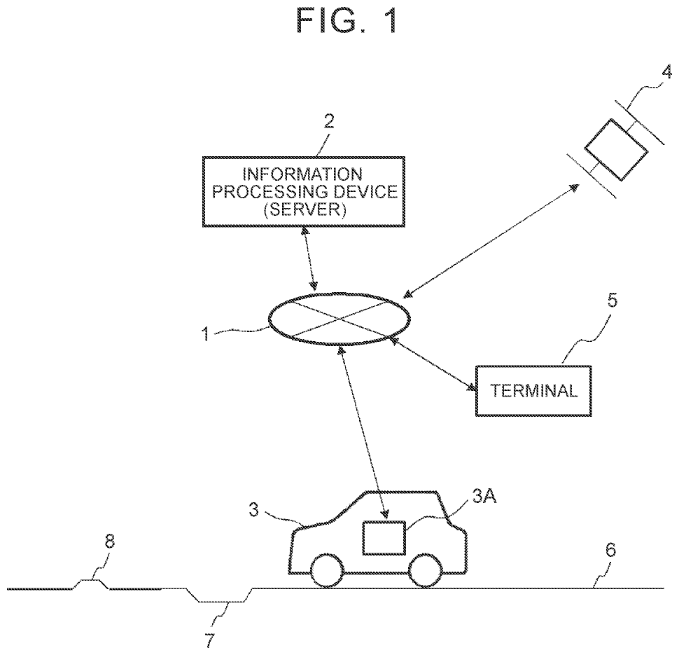

FIG. 1 is a diagram illustrating a configuration example of an information processing system according to an embodiment; In FIG. 1, the information processing system includes a network 1, an information processing device (server) 2, an in-vehicle terminal 3A mounted on the vehicle 3, satellites 4, and a terminal 5.

The network 1 is a public network such as the Internet. The information processing device 2 can communicate with the in-vehicle terminal 3A, the artificial satellites 4, and the terminal 5 mounted on the vehicles 3 via the network 1. The in-vehicle terminal 3A transmits information on the unevenness of the road surface of the road 6 on which the vehicle 3 travels via the network 1 (referred to as unevenness-related information) to the information processing device 2.

The information processing device 2 identifies a place where a predetermined unevenness is generated on the road 6 based on the unevenness-related information received from the vehicle 3. The unevenness of the road surface is, for example, a depression 7 formed in the road surface of the road 6 or a predetermined (detection target) unevenness such as the elevation 8. The predetermined unevenness is an unevenness to be observed by the artificial satellite 4. For example, the predetermined unevenness is an unevenness having a variation amount (height) to the extent that inspection or repair is necessary. It is possible to appropriately set what kind of unevenness is to be a predetermined unevenness and a detection method thereof.

The unevenness-related information includes information for specifying a place where a predetermined unevenness is generated by the information processing device 2. For example, the unevenness-related information may include information indicating the rotational speed of each wheel (for example, four wheels if four wheels) of the vehicle 3 (referred to as rotational speed information), information indicating the vehicle speed of the vehicle 3 (referred to as vehicle speed information), information indicating the position of each wheel (referred to as wheel position information), and the like. The wheel position information may include information indicating the size of each tread and wheel base between the front and rear wheels of the vehicle 3 and the wheels. The wheel position information can be used as information indicating the position of the vehicle 3. However, information indicating the position of the vehicle 3 other than the wheel position information may be acquired by the information processing device 2.

The artificial satellite 4 is, for example, an artificial satellite equipped with a synthetic aperture radar (SAR). In accordance with an instruction (command) from the information processing device 2, the artificial satellite 4 generates image data obtained by observing an area including a place (road 6) where a predetermined unevenness based on the unevenness-related information specified by the information processing device 2 is generated. The image data is data indicating the terrain of the corresponding area. It is possible to observe a topographical displacement at a place where a predetermined unevenness based on the unevenness-related information is generated by the image data. The artificial satellite 4 transmits image data to the information processing device 2 via the network 1.

The image data is an example of “data observed by an artificial satellite” (referred to as observation data). However, the data observed by the artificial satellite 4 may be other than the image data. The artificial satellite 4 may generate observation data using an optical sensor other than the SAR or a microwave sensor.

The information processing device 2 transmits the image data to the terminal via the network 1. However, as an option, the information processing device 2 may analyze the image data, and the information processing device 2 may transmit the image data to the terminal 5 when the information processing device 2 determines that a predetermined unevenness has occurred even in the analysis result. In this case, when the predetermined unevenness based on the unevenness-related information is erroneous detection of the unevenness, it is possible to avoid transmitting the image data to the terminal 5.

The terminal 5 is a terminal 5 for road management used by an administrator of the road 6. The terminal 5 can display an image based on the image data. The administrator of the road 6 can inspect the unevenness with reference to the image. As a result, it is possible to save the time and effort of the actual inspection. In addition, it is possible to determine whether or not to actually look at the unevenness.

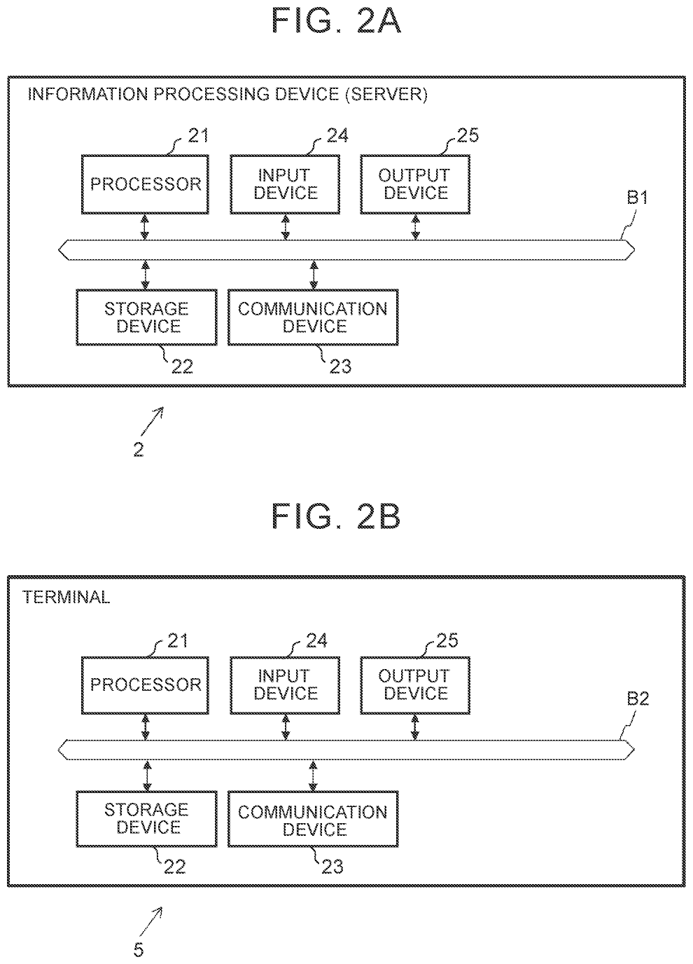

FIG. 2A is a diagram illustrating a configuration of an information processing device 2. FIG. 2B is a diagram illustrating an exemplary configuration of a terminal 5. The information processing device 2 and the terminal 5 are general-purpose or special-purpose computers such as a personal computer (PC), a workstation (WS), or a server machine. The information processing device 2 and the terminal 5 may be fixed terminals or portable terminals. The portable terminal is a laptop PC, a smart device (such as a smartphone and a tablet terminal), or the like. However, the portable terminal is not limited to the example. The information processing device 2 may be one information processing apparatus or a set of two or more information processing devices (cloud).

In FIG. 2A, the information processing device 2 includes a processor 21 as a processing unit or a control unit (controller), a storage device 22, a communication device 23, an input device 24, and an output device 25, which are connected to each other via a bus B1.

The storage device 22 includes a main storage device and an auxiliary storage device. The main storage device is used as a storage area for programs and data, a deployment area for programs, a work area for programs, and a buffer area for communication data. The main storage device consists of a Random Access Memory (RAM) or a combination of RAM and Read Only Memory (ROM). The auxiliary storage device is used as a storage area for data and programs. Non-volatile storage media such as hard disks, Solid State Drive (SSDs), flash memories, and EEPROM (Electrically Erasable Programmable Read-Only Memory, for example, can be used as the secondary storage device.

The communication device 23 is a circuit that performs communication processing. The communication device 23 operates as a transmission unit and a reception unit (communication unit). For example, the communication device 23 is a network interface card (NIC). Further, the communication device 23 may be a wireless communication circuit that performs wireless communication (LTE, 5G, wireless LAN (Wi-Fi), BLE, etc.). The communication device may be a combination of a NIC and a wireless communication circuit.

The input device 24 includes keys, buttons, a pointing device, a touch panel, and the like. The input device 24 is used to input information. The input device 24 may include a microphone (voice input device). The output device 25 is, for example, a liquid crystal display or an organic EL display. The output device 25 displays information and data. The output device 25 may include a speaker (audio output device).

The processor 21 is, for example, a Central Processing Unit (CPU). The processor 21 performs various processes by executing various programs stored in the storage device 22. For example, the processor 21 executes a process of acquiring the unevenness-related information from the in-vehicle terminal 3A of the vehicle 3 and a process of specifying a place where a predetermined unevenness is generated. Further, the processor 21 executes a process of transmitting an instruction for observing the specified location to the artificial satellite 4, a process of receiving observation data (image data) from the artificial satellite 4, a process of transmitting image data to the terminal 5, and the like.

Similarly to the information processing device 2, the terminal 5 includes a processor 21 connected to the bus B2, a storage device 22, a communication device 23, an input device 24, and an output device 25. However, devices having different specifications or performance may be applied depending on the purpose of processing or the magnitude of the load. The processor 21 of the terminal 5 performs display control of the output device 25 (display) and executes processing of displaying an image based on the image data on the display.

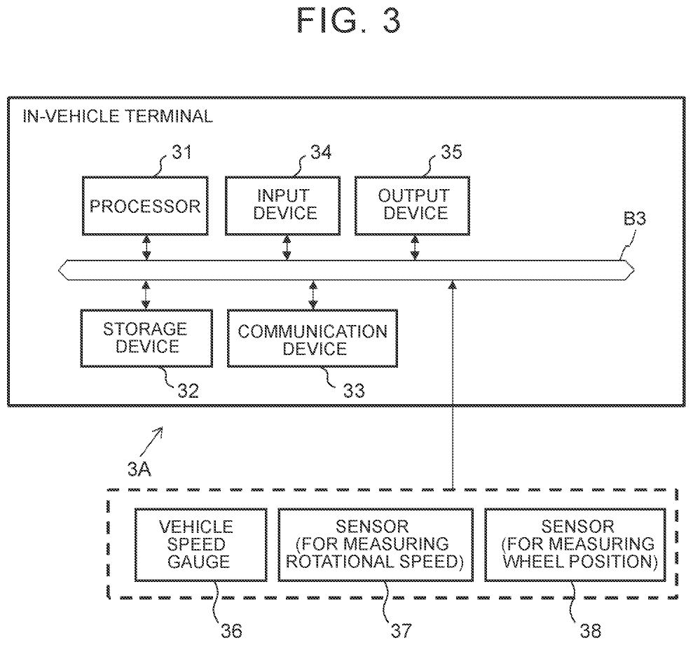

FIG. 3 is a diagram illustrating an exemplary configuration of an in-vehicle terminal 3A. The in-vehicle terminal 3A includes a processor 31 connected to a bus B3, a storage device 32, a communication device 33, an input device 34, and an output device 35. The processor 31, the storage device 32, the communication device 33, the input device 34, and the output device 35 may be the same as those of the processor 21, the storage device 22, the communication device 23, the input device 24, and the output device 25, respectively. However, the specifications and performance of the same type of apparatus differ depending on the application and load. The communication device 33 is constituted by a wireless communication circuit.

The in-vehicle terminal 3A receives information/data from the vehicle speed gauge 36, the sensor 37, the sensor 38, and the like mounted on the vehicle 3. The vehicle speed gauge 36 measures a vehicle speed that is a speed of the vehicle 3. For example, the vehicle speed gauge 36 calculates the vehicle speed from the rotational speed of the output shaft of the transmission measured by the sensor.

The sensor 37 is a sensor that measures the rotational speed of each wheel (four wheels, two in front and two in rear in the present embodiment) included in the vehicle 3. The sensor 38 measures wheel position information indicating a position of each wheel included in the vehicle 3 on the ground surface. The wheel position information can be acquired by calculating the position of the wheel on the ground surface based on the current position and the direction of the vehicle 3 and the sizes of the tread, the wheel base, and the wheels of the vehicle 3. The present position and the direction (traveling direction) of the vehicle 3 can be acquired using, for example, a Global Positioning System (GPS) function included in the vehicle 3. As a method of measuring the rotational speed and a method of acquiring the wheel position information, any existing method can be applied.

The processor 31 of the in-vehicle terminal 3A executes the above-described process of acquiring the information indicating the vehicle speed, the wheel position information, and the information indicating the rotational speed of the wheel by executing the program stored in the storage device 32. Then, the processor 31 generates the unevenness-related information including the acquired information, and executes a process of transmitting the unevenness-related information to the information processing device 2 via the network 1.

Note that a plurality of CPUs may be used as the above-described processors 21 and 31, or a multi-core CPU may be used. At least some of the processes performed by the CPU may be performed by processors other than the CPU, such as Digital Signal Processor (DSPs) or Graphical Processing Unit (GPUs). Further, at least a part of the processes performed by the CPU may be performed by a dedicated or a general-purpose integrated circuit (hardware). Integrated circuitry may include Application Specific Integrated Circuit (ASIC), or Field Programmable Gate Array (FPGA). Alternatively, at least a part of the processes performed by the CPU may be executed by a combination of the processor and the integrated circuit. Combinations are referred to as, for example, microcontrollers (MCUs), System-on-a-chip (SoCs), system LSIs, or chipsets.

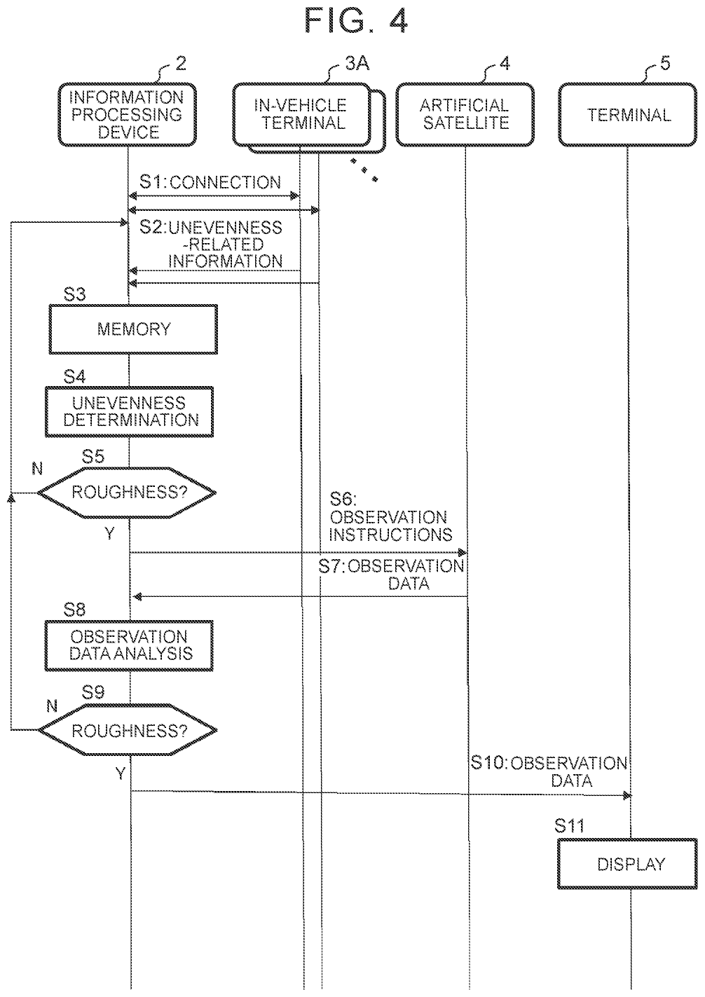

FIG. 4 is a sequence diagram illustrating an operation example of the information processing system. The information processing device 2 establishes a communication line with one or more in-vehicle terminal 3A (step S1). Each of the in-vehicle terminal 3A transmits the unevenness-related information to the information processing device 2. The unevenness-related information includes at least vehicle speed information, rotation speed information, and wheel position information. The transmission timing of the unevenness-related information can be set as appropriate. For example, the in-vehicle terminal 3A may transmit the unevenness-related information in response to a request from the information processing device 2, or the in-vehicle terminal 3A may periodically or periodically transmit the unevenness-related information.

The processor 21 of the information processing device 2 stores the unevenness-related information received from each of the in-vehicle terminal 3A in the storage device 22 (step S3). The storage device 22 is an example of a “storage unit”.

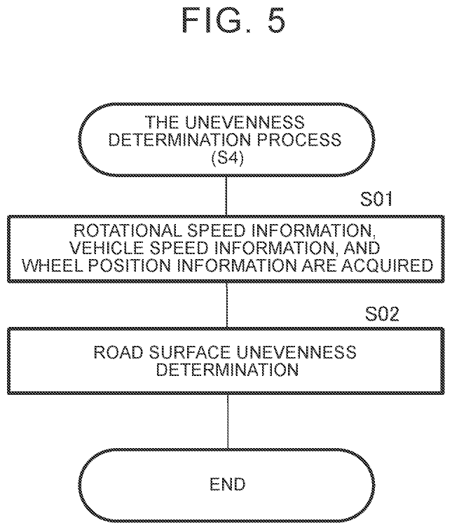

The processor 21 of the information processing device 2 performs an unevenness determination process (step S4). FIG. 5 is a flowchart illustrating an example of the unevenness determination process. In step S01, the processor 21 of the information processing device 2 acquires vehicle speed information, rotation speed information, and wheel position information. In the present embodiment, the processor 21 of the information processing device 2 reads the vehicle speed information, the rotation speed information, and the wheel position information from the storage device 22.

In step S02, as the road surface unevenness determination process, the processor 21 of the information processing device 2 performs the following abnormality detection on the road surface. That is, the processor 21 detects a malfunction (abnormality) of the road 6 on which the vehicle 3 is traveling based on the unevenness-related information received from each vehicle 3. Specifically, the processor 21 calculates the estimated rotational speed of the wheel based on the vehicle speed indicated by the vehicle speed information included in the unevenness-related information and the size of the wheel.

Then, the processor 21 detects a malfunction of the road 6 based on the estimated rotation speed and the rotation speed information. At this time, when the difference between the estimated rotational speed and the rotational speed indicated by the rotational speed information exceeds the predetermined first threshold value, the processor 21 detects that there is an abnormality (a predetermined unevenness) on the road 6. Alternatively, when the difference between the rotational speeds of the plurality of wheels provided in the vehicle 3 exceeds a predetermined second threshold value, the processor 21 detects that there is an abnormality (a predetermined unevenness) in the road 6. Then, the processor 21 specifies the position of the ground surface indicated by the wheel position information when the abnormality is detected as the position information of the “portion where the predetermined unevenness is generated” (referred to as the unevenness generation portion).

Note that the predetermined unevenness may be detected as follows. That is, the processor 21 calculates an estimated delay time from when the front wheel of the vehicle 3 passes to when the rear wheel passes, using the vehicle speed and the length of the wheel base. After the estimated time has elapsed, if there is a change in the rotational speed of the wheel that is not due to a similar change in the vehicle speed for the rear wheel and the front wheel, the processor 21 can determine that the wheel has fallen from the step or has risen from the step. Lowering or raising the step means that there are irregularities (depressions 7 or ridges 8). Then, the processor 21 calculates the difference between the estimated rotational speed of the wheel and the actual rotational speed as the variation amount, and converts the variation amount into the height of the step. Then, when the height (absolute value) of the step is larger than the threshold value, the processor 21 determines that the position where the step is detected is a position having a predetermined unevenness.

Returning to FIG. 4, the processor 21 of the information processing device 2 determines the presence or absence of a predetermined unevenness (step S5). Here, when the processor 21 detects that there is an abnormality in the road 6 in step S4, it determines that there is a predetermined unevenness.

Then, the processor 21 of the information processing device 2 transmits an observation instruction (command) of the unevenness generation portion including the position information of the unevenness generation portion to the artificial satellite 4 (step S6). In response to an observation instruction from the information processing device 2, the artificial satellite 4 generates observation data for an area including the position of the uneven portion. That is, the artificial satellite 4 generates image data (data observed by the artificial satellite 4, that is, observation data) indicating the shape of the ground surface of the area by using the SAR, and transmits the observation data to the information processing device 2 (step S7).

The information processing device 2 analyzes the observation data received from the artificial satellite 4, that is, the image data (step S8), and determines whether or not there is actually an unevenness such as the depression 7 or the elevation 8 in the unevenness generating portion (step S9). When it is determined that there are irregularities (Y in step S9), the information processing device 2 transmits the image data (corresponding to the image data obtained by visualizing the data observed by the artificial satellite) to the terminal 5 (step S10). In the transmission to the terminal 5, the image data from the artificial satellite 4 may be appropriately modified for suitable visualization. Steps S8 and S9 are optional.

In the terminal 5 that has received the image data, the processor 21 of the terminal 5 executes a display process of displaying an image based on the image data, that is, an image of the uneven portion on the output device 25 (display). As a result, the administrator of the road 6 can refer to the image of the uneven portion. That is, the administrator of the road 6 can inspect the road 6 using the image. At this time, if the road surface condition is clear from the image, it is unnecessary for the administrator of the road 6 to go to the site for visual recognition. That is, it is possible to save the time and effort for the administrator of the road 6 to actually visually recognize the unevenness generating portion. Alternatively, even if the administrator of the road 6 goes to all of the unevenness generating portions, it can be used as information for determining the priority order to go.

According to the present embodiment, the processor 21 (control unit) of the information processing device 2 specifies a place where a predetermined unevenness is generated based on the unevenness-related information (information on the unevenness of the road surface) acquired from the vehicle 3. Then, the information processing device 2 provides data (observation data) obtained by observing a portion where a predetermined unevenness is generated by the artificial satellite 4.

As described above, in the embodiment, the observation data of the artificial satellite 4 is provided based on the information on the unevenness of the road surface acquired from the vehicle 3. The processor 21 of the information processing device 2 provides observation data corresponding to a place where a predetermined unevenness is generated, so that it is possible to save time and effort for a person to actually check the unevenness of the road surface and go to the site. In addition, it is possible to prevent the inspection from becoming useless when the detection of the uneven portion is erroneous detection.

The information processing device 2 can transmit the data observed by the artificial satellite 4 to the terminal 5 for management of the road 6. As described above, by sending the observation data to the terminal 5 of the administrator of the road 6, the administrator can effectively utilize the observation data and reduce the number of cases in which the administrator actually sees the road.

Further, the information processing device 2 acquires information on the rotational speed of the wheels of the vehicle 3 and position information (wheel position information) of the vehicle 3 as information on the unevenness of the road surface. The rotational speed of the wheels when the vehicle is traveling is transmitted from the vehicle. Therefore, the information processing device 2 can easily acquire the rotation speed of the wheel. There is a correlation between the rotational speed of the wheel and the unevenness of the road surface. Therefore, the information processing device 2 can determine whether or not a predetermined unevenness is generated based on the rotational speed of the wheel. Further, the information processing device 2 can specify a point where a predetermined unevenness is generated from the wheel position information.

Further, the information processing device 2 can transmit a command to the artificial satellite 4 so as to observe a predetermined region including a portion where a predetermined unevenness is generated. The predetermined region is a geographical region including the road 6 where an abnormality is detected (unevenness occurrence portion), and is a region that can be observed (imaged) by the artificial satellite 4. The geographical area may be defined as, for example, an area in which the ground surface is divided into a rectangle, a regular hexagon, or a regular octagon. The information processing device 2 can operate the artificial satellite 4 by transmitting a command to the artificial satellite 4, and can acquire desired data. In addition, it is possible to suppress unnecessary acquisition of data.

Further, as a result of the information processing device 2 analyzing the data observed by the artificial satellite 4 (such as data captured by the artificial satellite 4), when the information processing device 2 determines that a predetermined unevenness is generated, the information processing device 2 may provide the data observed by the artificial satellite 4. Only when the information processing device 2 detects the predetermined unevenness using the observation data, the information processing device 2 provides the data observed by the artificial satellite 4, so that it is possible to suppress the provision of unnecessary data to the terminal 5.

By using an artificial satellite equipped with SAR as the artificial satellite 4, it is possible to obtain an appropriate image of the ground surface regardless of weather such as when there is a cloud. Further, the processor 21 of the information processing device 2 can provide the terminal 5 with image data obtained by visualizing the data observed by the artificial satellite 4. By providing the image data, it is possible to improve the convenience of the administrator.

As described in the operation example, the information processing device 2 can use a plurality of vehicles 3 each equipped with an in-vehicle terminal 3A for collecting (acquiring) the unevenness-related information. Then, the information processing device 2 can store information on the unevenness of the road surface acquired from the plurality of vehicles 3 in the storage device 22 (storage unit). By using the information from the plurality of vehicles 3, the information processing device 2 can efficiently detect the unevenness generation portion. It should be noted that the in-vehicle terminal 3A of the vehicle 3 may be configured to detect the unevenness generating portion and send information indicating the unevenness generating portion to the information processing device 2.

The above-described embodiment is merely an example, and the present disclosure may be appropriately modified and implemented without departing from the scope thereof. For example, the processes and means described in the present disclosure can be freely combined and implemented as long as no technical contradiction occurs. Further, the processes described as being executed by one device may be shared and executed by a plurality of devices. Alternatively, the processes described as being executed by different devices may be executed by one device. In the computer system, it is possible to flexibly change the hardware configuration (server configuration) for realizing each function.

The present disclosure can also be implemented by supplying a computer with a computer program that implements the functions described in the above embodiments, and causing one or more processors of the computer to read and execute the program. Such a computer program may be provided to the computer by a non-transitory computer-readable storage medium connectable to the system bus of the computer, or may be provided to the computer via a network. Non-transitory computer-readable storage media include, for example, any type of disk, such as a magnetic disk (floppy disk, hard disk drive (HDD), etc.), an optical disk (CD-ROM, DVD disk, Blu-ray disk, etc.). The non-transitory computer readable storage medium also includes a read only memory (ROM), a random access memory (RAM), an EPROM, an EEPROM, a magnetic card, a flash memory, an optical card, and any other type of media suitable for storing electronic instructions.

What is claimed is:

1. An information processing device comprising a control unit that:
identifies a portion where predetermined unevenness is generated based on information on unevenness of a road surface, the information being acquired from a vehicle; and
provides data observed by an artificial satellite, the data being data of the portion where the predetermined unevenness is generated.

2. The information processing device according to claim 1, wherein the control unit transmits the data observed by the artificial satellite to a terminal for road management.

3. The information processing device according to claim 1, wherein the control unit acquires information on a rotational speed of a wheel of the vehicle and position information of the vehicle as the information on the unevenness of the road surface.

4. The information processing device according to claim 1, wherein the control unit transmits a command to the artificial satellite such that the artificial satellite observes a predetermined area including the portion where the predetermined unevenness is generated.

5. The information processing device according to claim 1, wherein the control unit provides the data observed by the artificial satellite when the control unit determines that the predetermined unevenness is generated as a result of analyzing the data observed by the artificial satellite.

6. The information processing device according to claim 1, wherein the artificial satellite is an artificial satellite mounted with a synthetic aperture radar.

7. The information processing device according to claim 1, wherein the control unit provides image data obtained by visualizing the data observed by the artificial satellite.

8. The information processing device according to claim 1, further comprising a storage unit that stores the information on the unevenness of the road surface, the information being acquired from a plurality of the vehicles.

9. An information processing method comprising:
identifying, by an information processing device, a portion where predetermined unevenness is generated based on information on unevenness of a road surface, the information being acquired from a vehicle; and
providing, by the information processing device, data observed by an artificial satellite, the data being data of the portion where the predetermined unevenness is generated.

10. The information processing method according to claim 9, further comprising transmitting, by the information processing device, the data observed by the artificial satellite to a terminal for road management.

11. The information processing method according to claim 9, wherein the information on the unevenness of the road surface includes information on a rotational speed of a wheel of the vehicle and position information of the vehicle.

12. The information processing method according to claim 9, further comprising transmitting, by the information processing device, a command to the artificial satellite such that the artificial satellite observes a predetermined area including the portion where the predetermined unevenness is generated.

13. The information processing method according to claim 9, wherein the information processing device provides the data observed by the artificial satellite when the information processing device determines that the predetermined unevenness is generated as a result of analyzing the data observed by the artificial satellite.

14. The information processing method according to claim 9, wherein the artificial satellite is an artificial satellite mounted with a synthetic aperture radar.

15. The information processing method according to claim 9, wherein the information processing device provides image data obtained by visualizing the data observed by the artificial satellite.

16. A program that causes a computer to execute:
a step of identifying a portion where predetermined unevenness is generated based on information on unevenness of a road surface, the information being acquired from a vehicle; and
a step of providing data observed by an artificial satellite, the data being data of the portion where the predetermined unevenness is generated.

17. The program according to claim 16, further comprising a step of transmitting the data observed by the artificial satellite to a terminal for road management.

18. The program according to claim 16, wherein the information on the unevenness of the road surface includes information on a rotational speed of a wheel of the vehicle and position information of the vehicle.

19. The program according to claim 16, further comprising a step of transmitting a command to the artificial satellite such that the artificial satellite observes a predetermined area including the portion where the predetermined unevenness is generated.

20. The program according to claim 16, wherein the step of providing the data observed by the artificial satellite is executed by the computer when it is determined that the predetermined unevenness is generated as a result of an analysis of the data observed by the artificial satellite.
